(19) United States
(12) Patent Application Publication
(10) Pub. No.: US 2017/0296578 A1
(43) Pub. Date: Oct. 19, 2017

(54) TREATMENT OF CANCER WITH HYPOCHLOROUS ACID

(71) Applicant: REALM THERAPEUTICS, INC., Malvern, PA (US)

(72) Inventors: Mark SAMPSON, Malvern, PA (US); Svetlana PANICHEVA, Malvern, PA (US); Cary SCHOCKEMOEHL, Malvern, PA (US); Ethan SOLOMON, Malvern, PA (US)

(21) Appl. No.: 15/486,487

(22) Filed: Apr. 13, 2017

Related U.S. Application Data
(60) Provisional application No. 62/322,977, filed on Apr. 15, 2016.

(51) Int. Cl. A61K 33/00; A61K 9/00; A61K 39/395; A61K 45/06; C07K 16/28; A61K 39/00

(52) U.S. Cl. CPC A61K 33/00; A61K 45/06; A61K 9/0019; C07K 16/2818; A61K 9/0014; A61K 39/3955; A61K 2039/545; C07K 2317/76

(57) ABSTRACT

The present invention relates to hypochlorous acid compositions and their use in therapy for cancer patients.

### CROSS REFERENCE TO RELATED APPLICATIONS

This application claims the benefit of U.S. Provisional Patent Application No. 62/322,977, filed Apr. 15, 2016, the entire contents of which are hereby incorporated by reference.

### FIELD OF THE INVENTION

The present invention relates to hypochlorous acid compositions and their use in therapy for cancer patients.

### BACKGROUND OF THE INVENTION

Despite major advances in cancer treatment, cancer remains one of the leading causes of death globally. Hurdles in designing effective therapies include cancer immune evasion, in which cancer cells escape destructive immunity, as well as the toxicity of many conventional cancer treatments such as radiation therapy and chemotherapy, and even newer treatments such as checkpoint inhibitors, where the body's inflammatory reaction to damaged tissue impacts the patient's ability to tolerate the therapy or impacts the efficacy of the treatment.

Accordingly, it is an object of this invention to improve cancer therapy by activating, modulating, controlling, and/or ameliorating various immunological and/or inflammatory responses, to both improve efficacy of therapeutic regimens and to manage the debilitating side effects of toxic therapies.

### SUMMARY OF THE INVENTION

In various aspects, the invention provides methods of treating cancer patients with hypochlorous acid compositions, optionally with one or more additional cancer therapies. In various embodiments, administration of the hypochlorous acid composition slows or inhibits the growth or progression of cancer, and/or prevents or ameliorates painful side effects of other therapies. By modulating inflammatory processes, the invention helps to control the growth, progression, and spread of cancer, including in combination with other agents, such as cancer immunotherapies. Further, by controlling the inflammatory response to damaged tissue, embodiments of the invention aid the body's repair mechanisms and/or prevent or reduce the painful side effects of primary cancer therapies that are harmful to non-cancer cells and tissues. The present invention in various embodiments improves cancer therapy by combining HOCl therapy with other cancer therapies, such as cancer immunotherapy (e.g., immune checkpoint inhibitor therapy), tumor resection, radiation therapy, and/or chemotherapy.

The hypochlorous acid composition comprises an effective amount of hypochlorous acid. While the amount can vary depending on the condition of the patient and/or route of administration to the patient, or to tissues or cells, the compositions in the various embodiments may comprise hypochlorous acid at from 10 to 400 μM, or from 400 to 1000 μM, or from 1 to 100 mM, or from 100 to 1000 mM. The hypochlorous acid composition can be administered topically or locally to malignant or damaged tissues, or in other embodiments, is administered topically to provide systemic effects to control cancer growth and progression. In some embodiments, the hypochlorous acid composition is administered systemically.

In some embodiments, the hypochlorous acid composition is administered to a patient before, during, or after treatment with immunotherapy, such as immune checkpoint inhibitor therapy. The HOCl composition, in combination with immune checkpoint inhibitor therapy, further slows the growth and/or progression of the tumor. In some embodiments, the combination may prevent or ameliorate side effects of the immunotherapy, such as, but not limited to, immune-related adverse events.

In other embodiments, the invention involves administering a hypochlorous acid composition to a patient undergoing a cancer therapy selected from resection (e.g., surgery), radiation therapy, chemotherapy, or a biologic therapy. In accordance with embodiments of the invention, the hypochlorous acid composition slows growth or progression of the tumor or cancer, and/or manages or ameliorates acute and/or delayed toxicity, which may manifest as one or more of nausea and vomiting, alopecia (hair loss), skin lesions or sores, dermatitis, mucosal lesions or sores (e.g., oral mucositis), and bone marrow depression. Hypochlorous acid treatment may further reduce or prevent damage to the nervous system, heart, lungs, liver, kidneys, gonads or other organs.

In some embodiments, the hypochlorous acid composition is administered to prevent or reduce (or reduce the severity of) skin or mucosal lesions or sores associated with the cancer therapy.

Other aspects and embodiments of the present invention will be apparent from the following detailed description.

### BRIEF DESCRIPTION OF THE DRAWINGS

FIG. 1 shows the mean tumor volume in mice treated with either anti-CTLA4 antibodies alone or in combination with HOCl topical gel. Group 1 mice received no treatment after transplantation of CT26 cells. Group 2 mice received anti-CTLA4 4F10 antibodies on Day 8, 11, and 14 of the study, while Group 3 received the same treatment supplemented with topical administration of HOCl gel twice daily starting on Day 3 of the study. Group 4 mice received anti-CTLA4 9H10 antibodies on Day 14, 17, and 20 of the study, while Group 5 received the same treatment supplemented with topical administration of HOCl gel twice daily starting when tumors reached 100 mm³ in size.

FIG. 2 shows the mean tumor volume in mice treated with either anti-CTLA4 antibody alone or in combination with HOCl topical gel. Group 1 received transplanted tumor cells and no further treatment. Group 2 received i.p. administration of anti-CTLA4 9H10 on days 12 (100 μg), 15 (50 μg), and 18 (50 μg) of the study. In addition to antibody treatment, group 3 received 200 mg of topical HOCl gel (500 ppm) twice daily (BID), starting on day 12 of the study. Group 4 received antibody treatment and 200 mg topical HOCl gel (1000 ppm) four times daily (QID).

### DETAILED DESCRIPTION OF THE INVENTION

The invention provides for methods of treating cancer patients with hypochlorous acid compositions, optionally with one or more additional therapies. In various embodiments, administration of the hypochlorous acid composition slows or inhibits tumor growth or cancer progression, and/or prevents or ameliorates painful side effects of other therapies. Specifically, cancer relies on the inflammatory machinery to progress and to metastasize. By modulating these inflammatory processes, the invention helps to control the progression and spread of cancer, including in combination with other agents, such as cancer immunotherapies. Further, by controlling the inflammatory response to damaged tissue, embodiments of the invention aid the body's repair mechanisms and/or reduce the painful side effects of primary cancer therapies that are harmful to non-cancer cells and tissues. The present invention in various embodiments improves cancer therapy by combining hypochlorous acid therapy with cancer immunotherapy (e.g., immune checkpoint inhibitors), tumor resection, radiation, and/or chemotherapy.

Hypochlorous acid (HOCl) is an oxidant that is produced by the human body's natural immune system. HOCl is generated as the final step of the Oxidative Burst Pathway, with large quantities of HOCl being released into phagocytic vesicles to destroy invading microorganisms. As described herein, it is considered that HOCl exhibits previously unrecognized effects on the immune system, including effects on expression, processing, and/or release of immune effector molecules, among other effects.

Cells directing the innate and adaptive immune response (which can include monocytes, macrophages, dendritic cells, Langerhans cells, fibroblasts, and keratinocytes, among others) secrete cytokines and other soluble factors that may include, for example, one or more of TNF, IFNγ, IL-1β, IL-2, IL-4, IL-6, IL-8, IL-10, IL-18. Cytokine release patterns vary, both between cytokines as well as cell types. For example, many immune mediators are secreted through classical secretory pathways including regulated or constitutive exocytosis or by degranulation. In classical secretory pathways, cytokines are translated with signal peptides in the endoplasmic reticulum (ER), trafficked in vesicles to the golgi complex, and subsequently to the cell surface for release. In the case of degranulation, cytokines and/or other cargo are stored in granules for later release. On the other hand, certain cytokines, such as IL-1β and IL-18, which are activated by the inflammasome and play a basic role in the initiation of inflammatory responses, are secreted via nonclassical secretory pathways. Specifically, these molecules are synthesized as inactive precursors, and once activated by caspase-1 cleavage, are potentially secreted either by membrane transporters, in exosomes or microvesicles, or perhaps even by cell lysis. See, for example, Lacy and Stow, Cytokine release from innate immune cells: association with diverse membrane trafficking pathways, Blood 118(1) (July, 2011). Further, innate and adaptive immune responses are regulated at the genomic level by NF-κB complex. Hypochlorous acid in accordance with various embodiments, can impact essential processes involved in the production of cytokines and immune effector molecules.

While the role of endogenous reactive oxygen species (ROS) in the inflammatory and immune processes has been somewhat clouded by conflicting data, ROS are generally considered as activators of the inflammasome. See, Harijith A, et al., Reactive oxygen species at the crossroads of inflammasome and inflammation, Front. Physiol. 5:352 (2014). For example, endogenously generated hypochlorous acid is often regarded as a pro-inflammatory molecule. See, Schieven G L et al., Hypochlorous acid activates tyrosine phosphorylation signal pathways leading to calcium signaling and TNFalpha production, Antioxid. Redox Signal 4(3):501-7 (2002); Pullar J L, et al., Living with a killer: the effects of hypochlorous acid on mammalian cells, IUBMB Life, 50(4-5):259-66 (2000). HOCl generation in vivo has been postulated to mediate inflammation in chronic inflammatory disease. Halliwell et al., Oxidants, inflammation, and anti-inflammatory drugs, FASEB 2:2867-2873 (1988). In contrast, the present disclosure shows that HOCl can have beneficial immunomodulation and therapeutic properties that find use in cancer therapy, for example, in cancer prevention and in cancer treatment.

In one aspect, the present invention provides a method for treating a cancer patient, comprising administering to a patient an effective amount of a hypochlorous acid composition. The hypochlorous acid composition can be administered topically or locally to malignant or damaged tissues, or in other embodiments, is administered topically to provide systemic effects to control cancer growth and progression. In some embodiments, the hypochlorous acid is administered topically to enhance or create synergistic effects in combination with another therapy applied topically or systemically. In some embodiments, the hypochlorous acid composition is administered systemically, alone or in combination with another therapy.

In another aspect, the present invention provides a method for treating pre-cancerous lesions. In some embodiments, the method prevents (or slows progression of) pre-cancerous lesions from developing into cancers. Exemplary pre-cancerous lesions include Actinic Keratosis.

As used herein, the term “treating” refers to providing therapy to a patient to slow or stop growth, progression or metastasis of a cancer, or to reduce or shrink established tumors, as well as in some embodiments, to prevent the onset, metastasis or re-occurrence of cancer, or to prevent or ameliorate side effects of other therapies.

As used herein, the terms “cancer” and “tumor” refer to an uncontrolled growth of cells that may interfere with the normal functioning of the bodily organs and systems, including both primary and metastatic cancers or tumors. Primary cancers or tumors that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastasis.

Illustrative cancers or precancerous conditions that may be treated in accordance with this disclosure include, but are not limited to, actinic keratosis, squamous cell carcinoma in situ (Bowen disease), squamous and basal cell carcinoma, melanoma, biliary tract cancer, bladder cancer, bone cancer, brain and central nervous system cancer, breast cancer, cancer of the peritoneum, cervical cancer, choriocarcinoma, colon or rectum cancer, connective tissue cancer, cancer of the digestive system, endometrial cancer, esophageal cancer, eye cancer, cancer of the head and neck, gastric cancer (including gastrointestinal cancer), glioblastoma, neuroblastoma, liver cancer (e.g., hepatic carcinoma), intra-epithelial neoplasm, kidney cancer, larynx cancer, leukemia, lung cancer (e.g., small-cell lung cancer, non-small cell lung cancer, adenocarcinoma of the lung, and squamous carcinoma of the lung), mesothelioma, myeloma, oral cavity cancer (lip, tongue, mouth, and pharynx), ovarian cancer, pancreatic cancer, prostate cancer, retinoblastoma, rhabdomyosarcoma, salivary gland carcinoma, testicular cancer, thyroid cancer, uterine cancer, vulval cancer, lymphoma including Hodgkin's and non-Hodgkin's lymphoma, Cutaneous T-Cell Lymphoma, B-cell lymphoma, as well as other carcinomas and sarcomas.

In various embodiments, the cancer is accessible for local or topical treatment with hypochlorous acid compositions, without systemic administration or invasive administration techniques. For example, in some embodiments the cancer is skin cancer (e.g., melanoma), breast cancer, lung cancer, testicular cancer, cervical cancer, uterine cancer, lymphoma, or lip or oral cancer.

In various embodiments, the cancer is stage I or II. In other embodiments, the cancer is stage III or IV. In some embodiments, the cancer is metastatic or non-metastatic melanoma.

In some embodiments, the hypochlorous acid composition is administered to a patient along with other cancer therapies. Without wishing to be bound by theory, it is believed that the co-administration of the hypochlorous acid composition and the additional cancer therapy produces synergistic therapeutic effects. For example, co-administration of the hypochlorous acid composition and the additional cancer therapy may act synergistically to reduce or eliminate the tumor or cancer, or slow the growth and/or progression of the tumor or cancer.

In some embodiments, the hypochlorous acid composition is administered to a subject before, during, or after treatment with other cancer therapies. In some embodiments, the hypochlorous acid composition is administered prior to administration of any other cancer therapies, for example, for prophylactic purposes.

In some embodiments, the hypochlorous acid composition is administered to a patient before, during, or after treatment with an immunotherapy. T cell activation plays an important role in tumor immunity and in autoimmune and inflammatory disorders. Specifically, activation of a naive T cell is initiated when T cell antigen receptors (TCR) recognize their specific antigen, such as an antigen present on tumor cells. Although TCR signal transduction is required for naive T cell activation, TCR activation alone is insufficient to generate an immune response. A secondary signal, known as co-stimulation, is needed for optimal activation of naive T cells. In particular, signal transduction through the TCR and a co-stimulatory receptor is required for full activation. Exemplary co-stimulatory receptors include CD28, 4-1BB, and OX-40.

Following activation, T cells transiently up-regulate the expression of a number of co-inhibitory receptors in order to suppress the immune response and limit the risk for autoimmune conditions. The co-inhibitory receptors (e.g., CTLA4 and PD-1) transduce inhibitory signals that counteract stimulatory signals and prevent overactivation of the immune system. However, by restricting the risk of self-recognition by the immune system, such immune checkpoints also limit the ability of the immune system to recognize unwanted antigens, such as tumor antigens. For example, cancer cells exploit this regulatory mechanism by continuously inducing co-inhibitory signals to evade immune destruction. Immune response to cancer cells can be restored through the use of immune checkpoint inhibitors that block these co-inhibitory signals.

In some embodiments, methods of the invention relate to the administration of a hypochlorous acid composition to a patient undergoing treatment with an immune checkpoint inhibitor. As used herein, an immune checkpoint inhibitor is any agent that disrupts, blocks, reduces, and/or inhibits the transmission of an immune inhibitory signal such as an immune co-inhibitory signal. In various embodiments, the immune checkpoint inhibitor is an agent that directly or indirectly, partially or completely, inhibits the activity of one or more molecules involved in the inhibitory (e.g., co-inhibitory) signaling pathway. Such inhibitory molecules include ligands and receptors, such as, without limitation, CTLA-4, PDL1, PDL2, PD1, BTLA, HVEM, TIM3, GALS, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), and various B-7 family ligands (including, but are not limited to, B7-1, B7-2, B7-DC, B7-H1, B7-H2, B7-H3, B7-H4, B7-H5, B7-H6 and B7-H7). For example, the immune checkpoint inhibitor can inhibit the inhibitory signaling pathways mediated by one or more of CTLA-4, PD-1 or PDL1, LAG3, TIM3, or KIR.

In some embodiments, the immune checkpoint inhibitor is a biologic therapeutic or a small molecule. In some embodiments, the immune checkpoint inhibitor is a biologic therapeutic and may comprise an antibody (e.g., a monoclonal antibody) or an antigen-binding portion thereof, or other ligand-binding molecule, and may be a mimic of the natural cellular receptor or ligand. In some embodiments, the immune checkpoint inhibitor is a small molecule. In other embodiments, the immune checkpoint inhibitor is an adnectin, an aptamer, an antisense polynucleotide, or a small interfering RNA.

In an exemplary embodiment, the immune checkpoint inhibitor comprises a monoclonal antibody (which may be humanized or fully human) that directly or indirectly, partially or completely, inhibits the inhibitory signaling pathways mediated by any of the inhibitory checkpoint molecules described herein. For example, the immune checkpoint inhibitor may comprise an antibody or antigen-binding portion thereof that blocks the activity of CTLA-4 or PD-1.

CTLA4 (Cytotoxic T-lymphocyte antigen 4) is a member of the immunoglobulin superfamily that is expressed exclusively on T-cells. CTLA4 acts to inhibit T-cell activation and is reported to inhibit helper T-cell activity and enhance regulatory T-cell immunosuppressive activity. It is thought that CTLA4 inhibits T cell activation by outcompeting CD28 in binding to CD80 and CD86, as well as actively delivering inhibitory signals to the T cell. Exemplary anti-CTLA4 antibodies or other ligands that may be used in accordance with embodiments of the present invention include, but are not limited to, ipilimumab/Yervoy (Bristol-Myers Squibb) and tremelimumab (Pfizer). Additional anti-CTLA4 antibodies include those disclosed in, WO1998/042752, WO2001/014424, WO2004/035607, WO2001/014424, WO2000/37504, US2005/0201994, US2002/0039581, US2002/086014, U.S. Pat. Nos. 5,811,097, 5,855,887, 6,051,227, 6,984,720, 6,682,736, 6,207,156, 5,977,318, 6,682,736, 7,109,003, and 7,132,281, and EP1212422, the entire disclosures of which are hereby incorporated by reference.

PD-1 (Programmed cell death protein 1) is a cell surface membrane protein of the immunoglobulin superfamily. One of the major roles of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD1 also acts to inhibit the TCR “stop signal” (Pardoll, 2012, Nature Reviews Cancer 12:252-264). PD1 is highly expressed on T_{reg} cells and may increase their proliferation in the presence of ligand (Pardoll, 2012, Nature Reviews Cancer 12:252-264). Exemplary anti-PD-1 antibodies or fusion proteins that may be used in accordance with embodiments of the present invention include, but are not limited to, pembrolizumab/Keytruda (Merck), nivolumab (BMS-936558, Bristol-Myers Squibb), AMP-224 (Merck), and pidilizumab (CT-011, Curetech Ltd.).

PD-L1 (Programmed cell death 1 ligand 1) is a ligand for PD-1, found on activated T cells, B cells, myeloid cells and macrophages. The complex of PD-1 and PD-L1 inhibits proliferation of CD8+ T cells and reduces the immune response. Exemplary anti-PD-L1 antibodies or fusion proteins that may be used in accordance with embodiments of the present invention include, but are not limited to, MDX-1105 (Medarex), MEDI4736 (Medimmune) MPDL3280A (Genentech), and BMS-936559 (Bristol-Myers Squibb).

Additional immune checkpoint inhibitors include, but are not limited to, anti-KIR antibodies, such as lirlumab (Innate Pharma) and IPH2101 (Innate Pharma); anti-B7-H3 antibodies, such as MGA271 (MacroGenics); and anti-LAG3 antibodies such as BMS-986016 (Bristol-Myers Squibb).

The HOCl composition, in combination with immune checkpoint inhibitor therapy, further slows the growth and/or progression of the tumor. In some embodiments, the combination may prevent or ameliorate side effects of the immune checkpoint inhibitor therapy, such as stomach pain, bloating, constipation or diarrhea, fever, breathing problems, or urinating problems. In some embodiments, the invention prevents or ameliorates immune-related adverse events, which can involve one or more of several organs or systems, such as skin, bowel, kidney, peripheral or central nervous system, liver, lymph nodes, eyes, pancreas, and lungs.

In some embodiments, methods of the invention provide for the administration of a hypochlorous acid composition to a patient undergoing treatment with an immune stimulating molecule. As used herein, the immune stimulating molecule is any agent that activates or enhances the transmission of an immune stimulatory signal such as an immune co-stimulatory signal. In various embodiments, the immune stimulating molecule comprises an agent that directly or indirectly, partially or completely, activates or enhances the activity of one or more molecules involved in the stimulatory (e.g., co-stimulatory) signaling pathway. Such stimulatory molecules include ligands and receptors, such as, without limitation, CD27, CD28, CD30, CD30 ligand, CD40, CD40 ligand, CD70, CD122, 4-1BB/CD137, CD137 ligand, OX40, OX40 ligand, GITR, GITR ligand, HVEM, LIGHT/CD258, CD80/B7-1, CD86/B7-2, TL1A, ICOS, and ICOS ligand. For example, the immune stimulatory molecule activates or enhances the stimulatory signaling pathway mediated by one or more of ICOS, GITR, OX40, CD137, CD122, or CD27.

In some embodiments, the stimulatory molecule is a biologic therapeutic or a small molecule. In some embodiments, the stimulatory molecule is a biologic therapeutic and may comprise an agonistic antibody or an antigen-binding portion thereof. In some embodiments, the stimulatory molecule is a small molecule agent. In some embodiments, the stimulatory molecule is an aptamer agonist.

In an exemplary embodiment, the stimulatory molecule comprises a monoclonal antibody (which may be humanized or fully human) or antigen-binding portion thereof that directly or indirectly, partially or completely, activates or enhances the stimulatory signaling pathway mediated by any of the stimulatory checkpoint molecules described herein. For example, the immune stimulatory molecule may comprise an agonist antibody that stimulates the activity of CD40, 4-1BB, OX40, GITR, CD80/B7-1, or CD86/B7-2. In another example, the stimulatory molecule may comprise a ligand (e.g., a soluble ligand) for any of the co-stimulatory receptors described herein such as a CD30 ligand, a CD40 ligand, an OX40 ligand, CD70, CD80/B7-1, CD86/B7-2, or LIGHT, or fragments or derivatives thereof.

CD27 is a co-stimulatory receptor that supports antigen-specific expansion of naive T cells and is vital for the generation of T cell memory. CD27 is also a memory marker of B cells. CD27 is activated by the transient availability of its ligand, CD70. In an embodiment, the present invention contemplates the use of an agonistic CD27 antibody, such as, without limitation, CDX-1127 (Celldex Therapeutics). In an embodiment, the present invention contemplates the use of the CD27 ligand (e.g., a soluble CD27 ligand), CD70 and/or fragments or derivatives thereof.

CD28 is a co-stimulatory receptor that is constitutively expressed on almost all human CD4+ T cells and on around half of all CD8 T cells. CD28 is activated by binding with its two ligands, CD80/B7-1 and CD86/B7-2. In an embodiment, the present invention contemplates the use of an agonistic CD27 antibody, such as, without limitation, TGN1112 or TGN1412 (TeGenero Immuno Therapeutics). In another embodiment, the present invention contemplates the use of agonistic antibodies of CD80/B7-1 or CD86/B7-2. In a further embodiment, the present invention contemplates the use of CD80/B7-1 and/or CD86/B7-2 (e.g., soluble CD80/B7-1 or CD86/B7-2) and/or fragments or derivatives thereof.

CD40 is a co-stimulatory receptor found on a variety of immune system cells including antigen presenting cells and activated CD4+ T cells. CD40 signaling is activated in the presence of the CD40 ligand. In an embodiment, the present invention contemplates the use of agonistic antibodies of CD40 such as, without limitation, CP-870,893 (Pfizer/VLST), dacetuzmumab (Seattle Genetics), and Chi Lob 7/4 (University of Southampton) as well as the agonistic anti-CD40 antibodies described in U.S. Pat. No. 6,843,989. The present invention further contemplates the use of the CD40 ligand (e.g., soluble CD40 ligand) and/or fragments or derivatives thereof.

CD122 is the interleukin-2 receptor beta sub-unit and is known to increase the proliferation of CD8+ effector T cells. In an embodiment, the present invention contemplates the use of NKTR-214 (Nektar Therapeutics), which is a CD122-biased immune-stimulatory cytokine.

4-1BB or CD137 is bound by CD137 ligand to induce T-cell proliferation. In an embodiment, the present invention contemplates the use of agonistic antibodies of 4-1BB/CD137 such as, without limitation, PF-05082566 (Pfizer) and urelumab/BMS-663513 (Bristol-Myers Squibb). The present invention further contemplates the use of the 4-1BB ligand (e.g., soluble 4-1BB ligand) and/or fragments or derivatives thereof.

OX40 is a co-stimulatory receptor that promotes the expansion of effector and memory T cells. This receptor is activated by the OX40 ligand. In an embodiment, the present invention contemplates the use of agonistic antibodies of OX40 such as, without limitation, MOXR0916/RG7888 (Genentech), and MEDI0562, MEDI6469, and MEDI6383 (MedImmune). The present invention further contemplates the use of the OX40 ligand (e.g., soluble OX40 ligand) and/or fragments or derivatives thereof.

GITR (Glucocorticoid-Induced TNFR family Related gene) is a co-stimulatory receptor that promotes T cells expansion including Treg expansion. GITR is activated by the GITR ligand expressed mostly on antigen presenting cells. In an embodiment, the present invention contemplates the use of agonistic antibodies of GITR such as, without limitation, TRX518 (GITR, Inc.) and MK-4166 (Merck). The present invention further contemplates the use of the GITR ligand (e.g., soluble GITR ligand) and/or fragments or derivatives thereof.

ICOS (Inducible T-cell costimulator) is expressed on activated T cells, and its ligand is ICOSL, which is expressed mainly on B cells and dendritic cells. In an embodiment, the present invention contemplates the use of agonistic antibodies of ICOS. The present invention further contemplates the use of the ICOS ligand (e.g., soluble ICOS ligand) and/or fragments or derivatives thereof.

In some embodiments, the patient receives therapy with engineered T cells. For example, autologous or donor T cells can be engineered to express a heterologous or engineered T cell receptor. In some embodiments, the engineered T cells express a chimeric antigen receptor (CAR), which may recognize CD19 in some embodiments. The engineered T cells may be administered to the patient by adoptive transfer. CAR-T technology is generally described in U.S. Pat. Nos. 7,446,179 and 6,410,319, which are hereby incorporated by reference in their entireties.

In some embodiments, methods of the invention provide for the administration of a hypochlorous acid composition to a patient undergoing treatment with a cancer vaccine. In some embodiments, the cancer vaccine may be a composition comprising one or more tumor antigens to stimulate an anti-tumor response, and alternatively may comprise antigen-pulsed dendritic cells or nanoparticle carriers. Exemplary tumor antigens and compositions comprising them are described in US 2010/0008920, which is hereby incorporated by reference in its entirety. In an embodiment, the cancer vaccine is a dendritic cell vaccine.

In other embodiments, the invention involves administering a hypochlorous acid composition to a patient undergoing a cancer therapy selected from resection (e.g., surgery), radiation therapy, chemotherapy, or a biologic therapy. Surgery involves the bulk removal of diseased tissues. While surgery is effective in removing localized tumors, it is not used, for example, in the treatment disseminated neoplastic conditions. Radiation therapy involves the exposure of living tissues to ionizing radiation causing death or damage to the exposed cells. However, both cancer cells and non-cancerous tissues are damaged by radiation. Chemotherapy involves the use of one or more compounds that inhibit cancer cell growth and is associated with significant toxicity to various organs.

Chemotherapy and radiation therapy are recognized to have both acute toxicity and delayed toxicity. Acute toxicity is manifested in side effects such as nausea and vomiting, fever, chills, abdominal pain, hyperglycemia, seizures, diarrhea, hypotension, ventricular arrhythmia, anaphylaxis, dermatitis, and localized phlebitis. Delayed toxicity can appear as bone marrow depression and concomitant immuno-suppression, renal damage, thrombosis, alopecia (hair loss), cataracts, liver damage, sterility, hemorrhagic cystitis, pulmonary edema, conjunctivitis, impotence, stomatitis, dermatitis, neurological defects, hypokalemia and hypocalcemia, and the like. Cutaneous reactions, hyperpigmentation and ocular toxicity have also been reported with virtually all non-hormonal anti-cancer drugs. In accordance with embodiments of the invention, the hypochlorous acid composition manages and/or ameliorates acute and/or delayed toxicity, which may include one or more of dermatitis, nausea and vomiting, alopecia (hair loss), skin lesions or sores, mucosal lesions or sores (for example, oral mucositis), and bone marrow depression. Hypochlorous acid treatment may further reduce or prevent damage to the nervous system, immune system, heart, lungs, liver, kidneys, gonads or other organs.

In some embodiments, the hypochlorous acid composition is administered to a patient before, during, or after, surgery or tumor resection. For example, the hypochlorous acid may be administered to the site of tumor resection, or may be administered before, during, or after tumor resection for a period of time to reduce the likelihood of recurrence, progression, or metastasis of remaining cancer cells or tissue. For example, the hypochlorous acid composition can be administered to the site of skin cancer (e.g., melanoma) resection, which may be administration at least once daily for a period of time (e.g., at least two weeks or at least one month). Alternatively, the hypochlorous acid composition can be administered topically (e.g., at least daily) for at least one month or at least two months, or more, after tumor tissue resection anywhere in the body.

In some embodiments, the hypochlorous acid composition is administered to a patient before, during, or after radiation therapy. Exemplary radiation therapy may include external beam therapy such as conventional external beam radiation therapy, steriotactic radiation including stereotactic radiosurgery and stereotactic body radiation therapy, 3-dimensional conformal radiation therapy, intensity-modulated radiation therapy, volumetric modulated arc therapy, particle therapy, Auger therapy, brachytherapy, intraoperative radiotherapy, and radioisotope therapy. For example, the hypochlorous acid composition may be administered to skin and/or mucosal surfaces to prevent and/or reduce (or reduce the severity of) lesions forming as a result of acute or delayed toxicity.

In some embodiments, the hypochlorous acid composition is administered to a patient before, during, or after chemotherapy or therapy with a biologic agent. In various embodiments, the patient is treated with one or more chemotherapeutic agents including, but not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A, and pharmaceutically acceptable salts, acids or derivatives of any of the above.

In some embodiments, the hypochlorous acid composition is administered to a patient before, during, or after treatment with one or more of kinase inhibitors, angiogenesis inhibitors, apoptosis inducers, topoisomerase (I or II) inhibitors, protease inhibitors, microtubule inhibitors, mitotic inhibitors, antimetabolites, signal transduction inhibitors, estrogen receptor inhibitors, EGFR inhibitors, Her2 inhibitors, aromatase inhibitors, monoclonal antibodies against tumor targets, anti-tumor vaccination, and immunotherapy.

In some embodiments, the hypochlorous acid composition is administered to a patient who is undergoing treatment with one or more of a kinase inhibitor. In an embodiment, the kinase inhibitor is a Janus kinase inhibitor. Exemplary Janus kinase inhibitors include, but are not limited to, ABT494, CYT387, Lestaurtinib, Pacritinib (SB1518), Ruxolitinib, Baracitinib, Filgotinib, and Tofacitinib.

In some embodiments, the hypochlorous acid composition is administered to a patient who is undergoing treatment with one or more of an anti-Her2/neu antibody such as Herceptin, an anti-EGFR antibody such as Erbitux, a growth factor receptor antibody such as Avastin, an anti-CD20 antibody such as Rituxan, or a small molecule inhibitor such as Tarceva, Iressa, or sunitinib. In still other embodiments, the patient is undergoing treatment with erlotinib, gefitinib, lapatinib, cetuximab, panitumumab, or imatinib.

In various embodiments, administration of the hypochlorous acid composition described herein prevents, delays, or reduces one or more side effects of treatment, rendering the treatment more effective, and increasing the likelihood that the patient can complete the recommended therapy. Administration of the hypochlorous acid composition in some embodiments reduces the need for breaks and/or interruptions in the therapy.

In some embodiments, the present invention prevents, delays, or reduces the oral, mucosal, and dermatological complications associated with cancer therapies. These side effects include, but are not limited to, scleroderma, erythema, edema, ulcerations, lesions, sores, and hyperkeratosis. Additional side effects include candidiasis, herpetic infections, deep fungal infections, bacterial infections, xerostomia, and oral mucositis (OM). Additional side effects include gastrointestinal side effects.

In an embodiment, administration of the hypochlorous acid composition provides for preventing, delaying, or reducing the severity of oral mucositis. Oral mucositis (OM), or stomatitis, is a common and debilitating complication of cancer chemotherapy and radiation therapy. OM is an inflammatory response of the oral mucosa and intraoral soft tissue structures in the oral cavity that occurs in response to the administration of radiation therapeutics and chemotherapeutics, as well as other cytotoxic therapies. It typically affects the inner surfaces of the cheeks and lips, the floor of the mouth, the lateral surfaces of the tongue and the bottom surfaces of the tongue and the soft palate. Lesions can also occur on the hard palate and upper surface of the tongue. Specifically, OM results from the systemic effects of stomatotoxic chemotherapy agents and from the local effects of radiation directed to the oral mucosa or the oral cavity. Mucositis can limit the patient's ability to tolerate the full regimen of chemotherapy or radiotherapy, thereby impacting the effectiveness of the treatment. Further, patients with damaged oral mucosa and reduced immunity resulting from chemotherapy and radiotherapy are also prone to opportunistic infections in the mouth. Administration of the hypochlorous acid composition may prevent, delay, or reduce the severity of the OM thereby allowing the patient to complete the planned course of therapy, by maintaining a sufficient nutritional state, and by avoiding the significant pain and discomfort associated with OM.

In an embodiment, the methods of the present invention contemplate the use of the hypochlorous acid composition for the prevention or delay of the onset of conditions and/or symptoms related to mucositis. Conditions related to mucositis vary from pain and discomfort to an inability to tolerate food or even fluids. In some embodiments, conditions related to OM can include erythema (reddening due to inflammation), swelling (edema), ulcerations, thickening of the keratin layer of the mucosa or skin (hyperkeratosis), a false membrane consisting of exudate and fibrin covering an ulceration (pseudomembranous mucosa), superficial infection caused by a yeast-like fungus of the genus Candida (candidiasis), swollen lymph nodes (lymphadenopathy), herpetic infections, deep fungal infections, bacterial infections, malnutrition (due to pain during eating), dehydration (due to pain during swallowing), bleeding (which can result in thrombocytopenia), the number and/or frequency of hospital and/or clinic visits due to OM, the need for breaks and/or interruptions in chemotherapy and/or radiation therapy and refusal of the radiation therapy and/or chemotherapy treatment regimen.

In various embodiments, administration of the hypochlorous acid composition provides for preventing, delaying, or reducing various skin conditions or symptoms associated with chemotherapy and/or radiation therapy. These include skin rashes, dry and itchy skin, sensitivity to light, burning and painful skin, hardening of skin (scleroderma), skin lesions, skin ulcers or sores, necrotic wounds, and malignant wounds.

In some embodiments, administration of a hypochlorous acid composition formulated for the eye, prevents, delays, or ameliorates toxic effects that manifest in the eye, which can include dry eye syndrome, conjunctivitis, cataracts, and eye itch.

In certain embodiments, the hypochlorous acid composition consists of essentially hypochlorous acid as the active agent, but in certain other embodiments may also contain other oxidizing or radical producing species such as a hypochlorite, hydroxide, H₂O₂ and O₃. In certain embodiments, the hypochlorous acid composition contains at least 50%, at least 60%, at least 70%, at least 80% hypochlorous acid relative to the total concentration of hypochlorous acid, hypochlorite, and molecular chlorine (Cl₂) (as 100%). The hypochlorous acid composition may have, however, at least 90%, at least 95%, or at least 98% hypochlorous acid relative to the total concentration of hypochlorous acid, hypochlorite, and molecular chlorine (Cl₂) (as 100%). Such embodiments may allow for higher levels of active chlorine to be administered, while avoiding any irritation as a result of the solution. Hypochlorite has been known for quite some time to have toxic properties on mammalian cells due to high pH in addition to required concentration of available chlorine, and thus may not be desirable, especially for long term use. Thus, in some embodiments, the level of hypochlorite in the solution or composition is limited (e.g., about 30% or less, or about 20% or less, or about 10% or less, or about 5% or less, or about 3% or less relative to the total concentration of hypochlorous acid, hypochlorite, and molecular chlorine (Cl₂) (as 100%).

The composition in various embodiments comprises hypochlorous acid at from 10 to 400 μM. In other embodiments, the composition comprises hypochlorous acid at 400 to 1000 μM. In still other embodiments, the composition comprises hypochlorous acid at from 1 to 100 mM, or from 10 to about 50 mM, or from 10 to about 25 mM. In still other embodiments, the composition comprises hypochlorous acid at from 100 to 1000 mM, or 100 to 500 mM, or 100 to 400 mM, or from 100 to 300 mM, or from 100 to 200 mM. In some embodiments, including for topical application, the composition comprises hypochlorous acid at from 10 to 40 mM, or at from 10 to 30 mM. Hypochlorous acid displays biologic effects that can be dose dependent.

In various embodiments, the hypochlorous acid composition may be administered to any tissue, organ, or parts of the body, and which may include the skin, mucus membranes, eyes, ears, nose, sinus cavity, throat, mouth (e.g., gingiva), lungs, connective tissue (including skeletal muscles, ligaments, tendons, joints), nervous system, intestinal tract (e.g., colon), urogenital system (including urinary tract or vagina), the peritoneum, or one or more organs such as the kidney, liver, or pancreas. In some embodiments, the route is chosen to administer the hypochlorous acid to the vicinity of the tumor or to tissues affected by the tumor or side effects of the therapeutic agent. The hypochlorous acid composition may be administered via any routes of administration which will range from via enteral (oral, gastric and rectal), parenteral (intravenous, intra-arterial, intraosseous, intra-muscular, intrathecal, sub-cutaneous) or other (sublingually, buccally, rectally, peritoneally, vaginally, intra-articular, by the ocular or otic route, nasally, cutaneously for topical or systemic effect, by inhalation or nebulization, or transdermally) or as an irrigant to one or more tissues or organs (e.g., during surgery). In an embodiment, the hypochlorous acid composition is administered via injection.

The composition may be formulated as a liquid, such as an eye drop, eye wash, gargle, mouth wash, nasal rinse, nasal spray, throat spray, or ear drop. In still other embodiments, the composition may take the form of a paste, cream, emulsion, gel, and/or foam for application (e.g., topical application) to the skin. Such formulations may be prepared using conventional additives known in the art and/or as described herein. Convenient applicators for creams, foams, and the like are known, and may be used in accordance with the present invention. Alternatively still, the composition may be formulated so as to be delivered by aerosol, mist, or steam (e.g., by nebulizer, and/or for pulmonary delivery), impregnated into dressings, adhesive, or dissolving strips, patches, suppositories, or encapsulated in silicon or other carriers, as nanoparticles or free-standing in liquids, suspensions, powders, pills or capsules, or as patches (e.g., transdermal patches and/or patches with micro-needles) for the purposes of release, targeted release or extended-release via enteral or parenteral administration.

Further still, the composition in various embodiments can be administered by aerosol to the lungs or by intravenous or subcutaneous delivery of, for example, particles that encapsulate and release HOCl in the circulation, either in a sustained manner or targeted to particular tissues or organs. For example, subcutaneous delivery may be achieved by the use of patches with micro-needles. In some embodiments, the solution or composition is formulated for colonic, vaginal, urinary tract, or peritoneal irrigation. In some embodiments, the solution or composition is formulated for irrigation of tissues or organs during or following surgery.

In some embodiments, the hypochlorous acid is administered topically, such as by a hydrogel composition. In some embodiments, the cancer involves the skin or is proximal to the skin (such as melanoma, basal cell carcinoma, squamous cell carcinoma, other cutaneous cancer, breast cancer, testicular cancer, or lip cancer). In some embodiments, the cancer therapy results in side effects that manifest in the skin, including, for example, lesions, sores, rash, blisters, hives, and itch. Administration of the topical hypochlorous acid composition reduces or prevents these conditions.

In embodiments related to the treatment of cancer, the hypochlorous acid composition is administered directly to the tumor or tissue or organ harboring the tumor. For example, in some embodiments, the hypochlorous acid composition is administered during surgical removal of the cancer. In other embodiments, the location of the tumor will instruct the most appropriate route of delivery, including by topical administration, pulmonary administration, colonic irrigation, catheter, or delivery to the oral cavity or sinus cavity. In some embodiments, the hypochlorous acid composition is administered topically, whether the cancer is cutaneous in origin, or proximal to the skin. In some embodiments, the hypochlorous acid provides systemic effects upon topical delivery, allowing for even remote cancers (such as liver, pancreatic, bladder, colorectal, and others) to be effectively treated using this route.

In embodiments related to the preventing, delaying, or reducing the side effects related to cancer therapy (e.g., chemotherapy or radiation therapy), the hypochlorous acid composition may be administered directly to the affected tissue or organ. For example, the hypochlorous acid composition may be directly applied to the oral mucosa, or to skin lesions or skin sores.

The hypochlorous acid composition of the invention may comprise a pharmaceutically acceptable carrier. Non-limiting examples of suitable carriers include hectorite, bentonite, laponite, oil emulsions, polyvinyl alcohol, povidone, hydroxypropyl methyl cellulose, poloxamers, carboxymethyl cellulose, hydroxyethyl cellulose, and purified water. The composition may also include various other ingredients, such as tonicity agents, buffers, surfactants, co-solvents, viscosity building agents, preservatives, and other therapeutic agents.

Regarding tonicity agents, such agents may be employed to adjust the tonicity of a composition, for example, in the case of an ophthalmic composition, to the tonicity of natural tears. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added and the type of composition. In general, however, the compositions will have a tonicity agent in an amount sufficient to cause the final composition to have an acceptable osmolality. For example, for an ophthalmic composition, the composition is generally in the range of about 150 to 450 mOsm, preferably 250 to 350 mOsm.

Regarding buffers, an appropriate buffer system (such as, for example, sodium phosphates, potassium phosphates, potassium carbonate, sodium bicarbonate, sodium borate or boric acid, phosphoric acid, or HCl) may be added to the compositions to prevent pH drift or loss of available free chlorine under storage conditions. The particular concentration will vary, depending on the agent employed. Preferably, however, the buffer will be chosen to maintain a target pH and/or level of hypochlorous acid.

Hypochlorous acid is highly unstable, a problem made more difficult when using higher strength solutions (e.g., above a few hundred ppm) as well as other formulation ingredients that can be destabilizing. Thus, in some embodiments, the composition includes a stabilizing amount of dissolved inorganic carbon (DIC) as disclosed in U.S. Pat. No. 8,871,278, which is hereby incorporated by reference in its entirety. For example, the composition may employ a stabilizing amount of DIC, which may be incorporated as a bicarbonate or carbonate salt of an alkali or alkaline earth metal, such as, for example, sodium, potassium, calcium, or magnesium. In some embodiments, the bicarbonates or carbonates are added prior to the formation of hypochlorous acid (e.g., by electrochemical treatment of saline or by enzymatic production), and in other embodiments, the bicarbonates or carbonates are added after production of the HOCl (e.g., after electrochemical treatment of saline or by enzymatic production). For example, the bicarbonate(s) or carbonate(s) may be contained in the precursor aqueous solution (e.g., water) or dry electrolyte, and/or incorporated in the electrolyzed solution or during formulation.

The DIC is incorporated at a “stabilizing amount,” which can be determined with reference to the change in the pH or HOCl content of the composition over time. Generally, the composition is considered stabilized if the amount of HOCl does not drop below about 75% of the initial value over a period of about 6 months. In certain embodiments, the HOCl content is stabilized for at least one year from the production date of the composition. Further, the stability of the composition may be determined with reference to the pH. Generally, the composition is considered stabilized if the pH does not vary by 1 unit over a period of about 6 months. In certain embodiments, the pH is stabilized for at least one year from the production date of the composition. The composition should be stored at 25° C. or at 20° C., or less for greater stability. 25° C. is the reference temperature for determination of stability. For stability testing, compositions are packaged in HDPE containers, stored in the dark, and kept unopened. The compositions may be stored at 4° C. until use in some embodiments.

The stabilizing amount of DIC (e.g., carbonate or bicarbonate) can be determined with reference to the HOCl content. For example, in certain embodiments, the stabilizing amount of the carbonate or bicarbonate is at a molar ratio of from about 5:1 to 1:5 with respect to the HOCl level, or from about 3:1 to about 1:2 with respect to the HOCl level. In some embodiments, the bicarbonates or carbonates are present in at least equal molar amounts with respect to the HOCl content. In still other embodiments, the DIC (e.g., bicarbonate or carbonate) is present at about 5:1, about 4:1, about 3:1, about 2:1, about 1:1, about 1:2, about 1:3, about 1:4, or about 1:5 with respect to HOCl content. In various embodiments, other buffering components such as phosphate buffers are also employed. For example, for compositions having HOCl from about 10 mM to about 30 mM, carbonate or bicarbonate may be present at an amount of from about 10 mM to about 75 mM to stabilize the formulation. In certain embodiments, the composition is for topical treatment of skin, and has HOCl in the range of 10 to 40 mM, and comprises sodium bicarbonate in the range of about 10 mM to about 75 mM, has a pH in the range of 5 to 7, and comprises a fluorosilicate (e.g., sodium magnesium fluorosilicate) at from 2 to 5% (e.g., about 3% or about 4%). In some embodiments, the composition is a hydrogel employing a silicate-based carrier such as sodium magnesium fluorosilicate, comprises sodium bicarbonate (e.g., from 10 mM to 50 mM) to stabilize the HOCl, and comprises phosphoric acid (and optionally sodium phosphate buffer) to target a slightly acidic pH (e.g., from 5 to 6.5). The composition may have a viscosity of from about 500 to about 50,000 cP, such as from about 1000 to about 40,000 cP, or from 1000 to about 30,000 cP. The composition in some embodiments has a conductivity of less than 10 mS/cm, such as from about 0.5 to about 5 mS/cm, such as from 0.5 to about 3 mS/cm, or about 1 or about 2 mS/cm in some embodiments.

Without being bound by theory, dissolved inorganic carbon (DIC), which generally includes carbonates, bicarbonates, carbonic acid and dissolved CO₂, provides low or minimal buffering capacity in the pH range targeted by the solutions and formulations described herein. Nevertheless, these solutions are effectively stabilized, such that the solutions and compositions are not dependent on “on-demand” production. The stabilizing effect can be due to, in-part, free radical scavenging ability of DIC to thereby slow the decomposition of HOCl.

The stabilized composition may be packaged for sale, using any suitable container, such as any suitable plastic or glass bottles, or bags, tubes, or cans (e.g., spray or aerosol). Certain container materials may provide advantages in shelf-life. In certain embodiments, the packaging material has minimal gas permeability (e.g., are non-permeable), including by species such as CO₂ and O₂. Thus, these containers maintain the stabilizing amount of dissolved inorganic carbon, without losing the stabilizer in the form of CO₂. The containers may be transparent, or may be opaque so that they are impenetrable by light.

Regarding a surfactant, various surfactants useful in conventional formulations may be employed. Exemplary surfactants include CREMOPHOR EL, lauramine oxide, myristyl dimethylamine oxide, polyoxyl 20 ceto stearyl ether, polyoxyl 40 hydrogenated castor oil, polyoxyl 23 lauryl ether and poloxamer 407.

Regarding viscosity building agents, such agents may be added to compositions of the present invention to increase the viscosity of the carrier. Examples of viscosity enhancing agents include, but are not limited to: synthetic silicates, polysaccharides, such as hyaluronic acid and its salts, chondroitin sulfate and its salts, dextrans, various polymers of the cellulose family; vinyl polymers; and acrylic acid polymers. For example, the composition may exhibit a viscosity of 1 to 400,000 centipoises (“cps”).

Regarding preservatives, no additional antimicrobial agent is required, since the HOCl will function as a preservative; however, in some embodiments HOCl is combined with a second preservative or antimicrobial agent such as silver. In various embodiments, the HOCl is manufactured as sterile.

In accordance with certain embodiments of the invention, the hypochlorous acid composition may be administered, for example, once daily or more than once daily. For example, the hypochlorous acid may be administered from about 1 to 10 times daily (e.g., about 2 times, 3 times, 4 times, 5 times, 6 times, 7 times, 8 times, 9 times, or 10 times daily). Alternatively the hypochlorous acid composition may be administered once a week or more than once a week (e.g., about 2 to 5 times per week). In some embodiments, the hypochlorous acid composition is administered topically from one to four times daily (e.g., twice daily).

Administration of the hypochlorous acid may be for any period of time as determined to be appropriate by, for example, an oncologist. In various embodiments, the hypochlorous acid composition is administered for at least one week, at least two weeks, at least three weeks, at least four weeks, at least five weeks, at least six weeks, at least seven weeks, at least eight weeks, at least nine weeks, at least ten weeks, at least eleven weeks, or at least twelve weeks. In some embodiments, the hypochlorous acid composition is administered for at least about one month, at least about two months, at least about three months, at least about four months, at least about five months, at least about six months, at least about seven months, at least about eight months, at least about nine months, at least about ten months, at least about eleven months, or at least about twelve months. In some embodiments, the hypochlorous acid is administered for at least about one year, or at least about two years, or more.

### Examples

### Example 1: Antitumor Effects of Hypochlorous Acid (HOCl) Gel Combined with Checkpoint Inhibitor Antibodies

A study was conducted to determine the efficacy of anti-CTLA4 antibodies (clones 4F10 and 9H10) alone or in combination with HOCl gel in a syngeneic CT26 colon carcinoma mouse model. Anti-CTLA4 4F10 and 9H10 antibodies were purchased from BioXcell (Lebanon, N.H.). HOCl gel containing 1,000 parts per million (ppm) available free chlorine (AFC) (approximately 14 mM HOCl) was prepared by formulating a solution of hypochlorous acid produced by electrolysis of sodium chloride.

CT26 colon carcinoma cells were initially grown in tissue culture and subsequently about 3×10⁵ cells were transplanted into the flank of 8-12 week old BALB/c mice. The caliper method was used to assess the ability of anti-CTLA4 antibodies alone or in combination with topical application of HOCl gel to inhibit tumor growth on a bi-weekly basis. Specifically, three treatment regimens were used. Group 1 received transplanted tumor cells and no further treatment. Groups 2 and 3 received i.p. administration of anti-CTLA4 4F10 on days 8 (100 μg), 11 (50 μg), and 14 (50 μg) of the study. In addition, group 3 received 200 mg of topical HOCl gel twice daily, starting on day 3 of the study. Groups 4 and 5 received i.p. administration of anti CTLA4 9H10 on days 14 (100 μg), 17 (50 μg), and 20 (50 μg) of the study. These treatment times were selected to coincide with tumor volume reaching approximately 100 mm³. In addition, group 5 received 200 mg of topical HOCl gel twice daily, also starting when tumor volume reached 100 mm³. Each treatment group included 10 BALB/c mice. Table 1 below summarizes the drug and treatment regimen for this study:

TABLE 1

| | Regimen 1 | | | | Regimen 2 | | | | Regimen 3 | | | |
|---|---|---|---|---|---|---|---|---|---|---|---|---|
| Group | Agent | μg/animal | Route | Schedule | Agent | μg/animal | Route | Schedule | Agent | μg/animal | Route | Schedule |
| 1 | No | | | | No | | | | No | | | |
| | treatment | | | | treatment | | | | treatment | | | |
| 2 | anti-CTLA | 100 | i.p. | Day 8 | anti-CTLA | 50 | i.p. | Days 11, | | | | |
| | 4F10 | | | | 4F10 | | | 14 | | | | |
| 3 | HOCl gel | 200 mg | tropical | BID × 28, | anti-CTLA | 100 | i.p. | Day 8 | anti-CTLA | 50 | i.p. | Days 11, |
| | | | | start on | 4F10 | | | | 4F10 | | | 14 |
| | | | | Day 3 | | | | | | | | |
| 4 | anti-CTLA | 100 | i.p. | Day 14 | anti-CTLA | 50 | i.p. | Days 17, | | | | |
| | 9H10 | | | (approx | 9H10 | | | 20 | | | | |
| | | | | 100 mm³ | | | | | | | | |
| | | | | tumor | | | | | | | | |
| | | | | volume) | | | | | | | | |
| 5 | HOCl gel | 200 mg | tropical | BID, start | anti-CTLA | 100 | i.p. | Day 14 | anti-CTLA | 50 | i.p. | Days 17, |
| | | | | at approx | 9H10 | | | (approx | 9H10 | | | 20 |
| | | | | 100 mm3 | | | | 10 mm³ | | | | |
| | | | | tumor | | | | tumor | | | | |
| | | | | volume | | | | volume) | | | | |

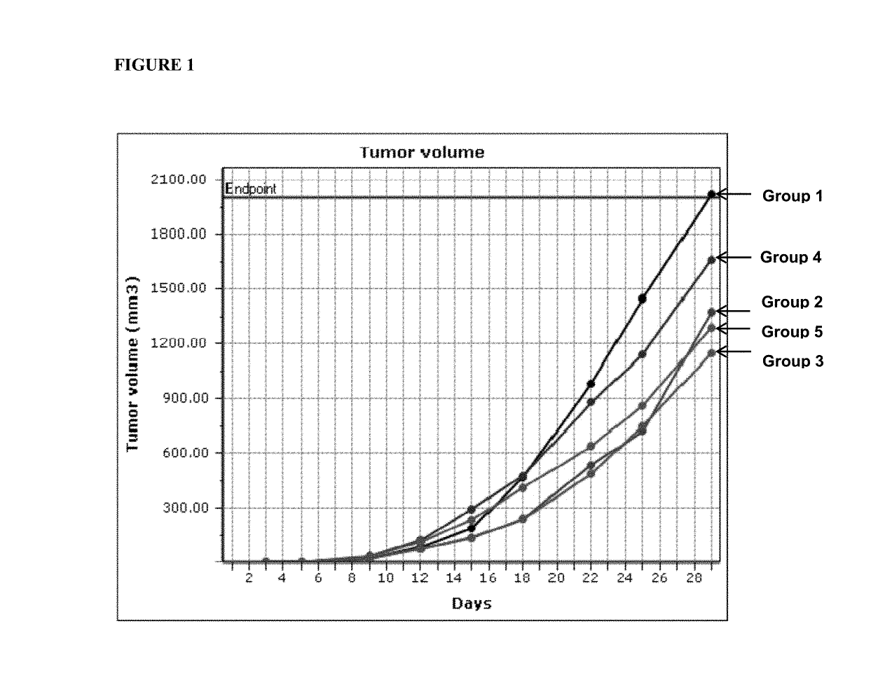

As shown in FIG. 1, all treatment groups exhibited a reduction of tumor volume compared to untreated controls. Data through day 29 of the study (FIG. 1) indicated that treatment using anti-CTLA4 4F10 alone reduced tumor volume by approximately 700 mm³ compared to untreated controls. The addition of twice daily topical administration of HOCl gel further reduced tumor volume by an additional 250 mm³ when compared to treatment anti-CTLA4 4F10 antibody alone. Treatment with anti-CTLA4 9H10 alone reduced tumor volume by 250 mm³ by day 29 compared to untreated controls. The addition of twice daily topical administration of HOCl gel (starting when tumors reached 100 mm³) further reduced tumor volume by approximately 500 mm³ compared to treatment with the anti-CTLA4 9H10 antibody alone. Notably, treatment with the combination of topical HOCl gel and anti-CTLA4 9H10 antibody starting on day 14 resulted in lower tumor volumes compared to treatment with anti-CTLA 4F10 started on Day 8 alone.

Altogether, these results indicate that treatment with HOCl in combination with either of the two anti-CTLA4 antibodies resulted in lower tumor volume compared with treatment with antibodies alone. Particularly, application of a combination therapy starting at Day 14 using HOCl gel and the anti-CTLA 9H10 clone resulted in lower tumor volume than treatment with anti-CTLA 9H10 alone or the combination of HOCl gel and anti-CTLA 4F10 when started on Day 3 of the study. These results suggest that topical HOCl application improves the efficacy of anti-CTLA4 antibodies (checkpoint inhibitors) in a synergistic fashion.

### Example 2: Antitumor Effects of Hypochlorous Acid (HOCl) Gel Combined with Checkpoint Inhibitor Antibodies

A study was conducted to further confirm the anti-tumor efficacy of the anti-CTLA4 antibody (clone 9H10) alone or in combination with HOCl gel in a syngeneic CT26 colon carcinoma mouse model. As described previously, the anti-CTLA4 9H10 antibody was purchased from BioXcell (Lebanon, N.H.). HOCl gel containing either 500 or 1000 parts per million (ppm) available free chlorine (AFC) was prepared by formulating a solution of hypochlorous acid produced by electrolysis of sodium chloride.

CT26 tumor cells were grown in tissue culture. Approximately 3×10⁵ CT26 cells were transplanted into the flank of 8-12 week old BALB/c mice. The ability of i.p. administration of anti-CTLA4, alone or in combination with topical application of HOCl gel, to inhibit the growth of tumors was assessed bi-weekly using the caliper method.

In this set of experiment, four different treatment regimens were utilized. Group 1 received transplanted tumor cells and no further treatment. Group 2 received i.p. administration of anti-CTLA4 9H10 on days 12 (100 μg), 15 (50 μg), and 18 (50 μg) of the study. In addition to antibody treatment, Group 3 received 200 mg of topical HOCl gel (500 ppm) twice daily (BID), starting on day 12 of the study. Group 4 received antibody treatment and 200 mg topical HOCl gel (1000 ppm) four times daily (QID). The day 12 initiation of treatment was selected to coincide with tumor volume reaching approximately 100 mm³. Each treatment group included 10 BALB/c mice. Table 2 below summarizes the drug and treatment regimen for this study:

TABLE 2

| | Regimen 1 | | | Regimen 2 | | | Regimen 3 | | |
|---|---|---|---|---|---|---|---|---|---|
| Group | Agent | μg/animal | Schedule | Agent | μg/animal | Schedule | Agent | μg/animal | Schedule |
| 1 | No | | | No | | | No | | |
| | treatment | | | treatment | | | treatment | | |
| 2 | anti-CTLA4 | 100 | i.p administration. | anti-CTLA4 | 50 | Days 15, | | | |
| | 9H10 | | Start on day 12 (tumors | 9H10 | | 18 i.p. | | | |
| | | | approx. 100 mm³) | | | | | | |
| 3 | HOCl gel | 200 | BID topical, start on | anti-CTLA4 | 100 | Day 12 | anti-CTLA4 | 50 | Days 15, |
| | (500 ppm) | | day 12 | 9H10 | | | 9H10 | | 18 i.p. |
| 4 | HOCl gel | 200 | QID topical, start on | anti-CTLA4 | 100 | Day 12 | anti-CTLA4 | 50 | Days 15, |
| | (1000 ppm) | | day 12 | 9H10 | | | 9H10 | | 18 i.p. |

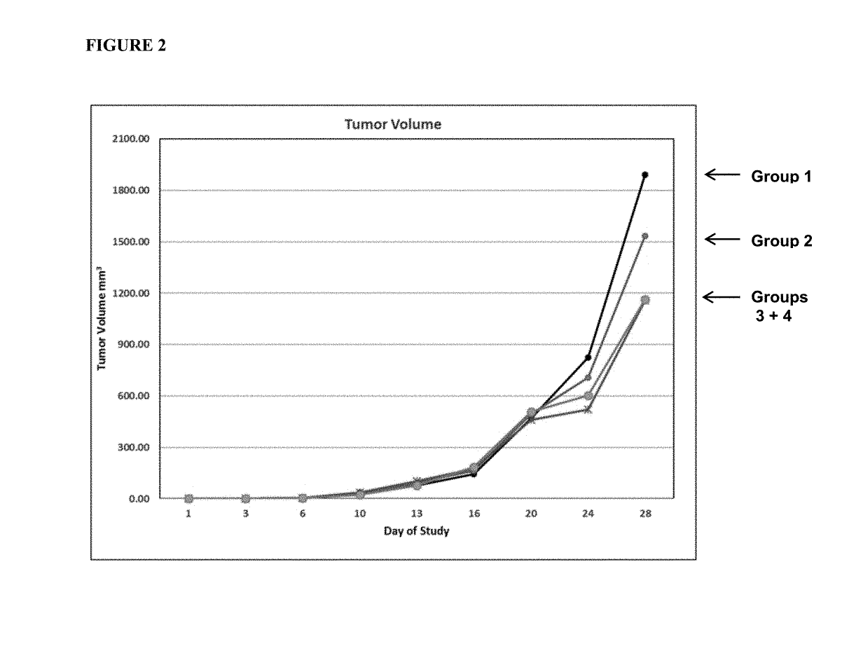

As shown in FIG. 2, all treatment groups exhibited a reduction of tumor volumes compared to the untreated controls. Data through day 28 of the study indicate that treatment using i.p. injection of anti-CTLA4 starting on day 12 of the study (i.e., group 2) successfully reduced tumor volume compared to untreated controls by approximately 350 mm³. The addition of twice daily topical administration of either 500 ppm or 1,000 ppm HOCl gel four times daily further reduced tumor volume by an additional 400 mm³ when compared to the anti-CTLA4 antibody alone.

Altogether, these results indicate that treatment with HOCl in combination with anti-CTLA4 antibody resulted in lower tumor volume compared with treatment with antibody alone. The results further suggest that topical HOCl improves the efficacy of anti-CTLA4 antibodies (checkpoint inhibitors) in a synergistic fashion.

Modifications of the disclosed embodiments incorporating the spirit and substance of the invention may occur to persons skilled in the art and such modifications are within the scope of the present invention.

All references cited herein are hereby incorporated by reference in their entirety.

1. A method for treating immune or inflammatory processes associated with cancer or cancer therapy in a patient, comprising administering to a patient an effective amount of a hypochlorous acid composition.

2. The method of claim 1, wherein the patient is undergoing a cancer therapy selected from cancer immune therapy, resection, chemotherapy, radiation therapy, or a combination thereof.

3. (canceled)

4. The method of claim 2, wherein the patient receives an immune checkpoint inhibitor that targets CTLA-4, PD-1 or PD-1L, LAG3, TIM3, or KIR.

5. The method of claim 4, wherein the immune checkpoint inhibitor is nivolumab, pembrolizumab, or ipilimumab.

6. The method of claim 2, wherein the patient receives an immune stimulating therapy that targets ICOS, GITR, OX40, CD137, CD122, or CD27.

7. (canceled)

8. The method of claim 1, wherein the patient is undergoing chemotherapy, radiation therapy, or a combination thereof.

9. The method of claim 8, wherein the patient suffers a side effect selected from skin rash, dry and/or itchy skin, hives, sensitivity to light, burning and painful skin, skin hardening (scleroderma), skin lesions, skin ulcers or sores, necrotic wounds, and malignant wounds.

10. The method of claim 8, wherein the patient suffers from mucosal lesions, which is optionally oral mucositis.

11. The method of claim 8, wherein the patient suffers from a side effect selected from dry eye syndrome, cataracts, conjunctivitis, and eye itch.

12-13. (canceled)

14. The method of claim 1, wherein the cancer is skin cancer, breast cancer, lung cancer, testicular cancer, cervical cancer, uterine cancer, lymphoma, thyroid cancer, lymphoma, sinus cancer, or lip or oral cancer.

15. (canceled)

16. The method of claim 1, wherein the cancer is prostate cancer, kidney cancer, brain cancer, ovarian cancer, colon cancer, bladder cancer, pancreatic cancer, stomach cancer, esophageal cancer, liver cancer, mesothelioma, parathyroid cancer, penile cancer, rectal cancer, or cancer of the small intestine.

17. A method for treating a pre-cancerous lesion, comprising, administering to a patient an effective amount of a hypochlorous acid composition to inhibit development of the lesion into cancer.

18. The method of claim 1, wherein the hypochlorous acid composition is administered topically.

19. The method of claim 1, wherein the hypochlorous acid composition is administered by injection, or by application to cancer-affected organs and/or tissues.

20-23. (canceled)

24. The method of claim 1, wherein the hypochlorous acid composition has from about 10 to about 100 mM HOCl.

25-26. (canceled)

27. The method of claim 1, wherein the hypochlorous acid composition is administered at least daily.

28. The method of claim 1, wherein the hypochlorous acid composition is administered for at least 4 weeks.
